(19) United States
(12) Patent Application Publication
(10) Pub. No.: US 2011/0091435 A1
(43) Pub. Date: Apr. 21, 2011

(54) PHYTOESTROGENIC FORMULATIONS FOR ALLEVIATION OR PREVENTION OF HAIR LOSS

(75) Inventors: Roberta Diaz Brinton, Rancho Palos Verdes, CA (US); Liqin Zhao, Los Angeles, CA (US)

(73) Assignee: University of Southern California

(21) Appl. No.: 12/911,999

(22) Filed: Oct. 26, 2010

Related U.S. Application Data
(63) Continuation-in-part of application No. 12/606,006, filed on Oct. 26, 2009.
(60) Provisional application No. 61/112,382, filed on Nov. 7, 2008.
(60) Provisional application No. 61/108,126, filed on Oct. 24, 2008.

(51) Int. Cl. A61K 31/352; A61P 5/24; A61K 38/43

(52) U.S. Cl. 424/94.1; 514/456

(57) ABSTRACT

Select phytoestrogen pharmaceutical compositions and methods of use for preventing or reducing one or more symptoms associated with hair loss or prostate cancer/prostate hypertrophy are described herein. These select phytoestrogen formulations are preferably composed only of two or more plant-derived estrogenic molecules and/or their structural analogues and exhibit binding preference to ERβ over ERα and agonist activity in non-reproductive tissues including brain.

### CROSS-REFERENCE TO RELATED APPLICATIONS

This application is a continuation-in-part of U.S. Ser. No. 12/606,006 entitled “Phytoestrogenic Formulations for Alleviation or Prevention of Menopausal Symptoms”, filed Oct. 26, 2009, which claims benefit of and priority to U.S. Ser. No. 61/112,382 entitled “Phytoestrogenic Formulations for Alleviation or Prevention of Neurodegenerative Disorders”, filed Nov. 7, 2008, and U.S. Ser. No. 61/108,126 entitled “Phytoestrogenic Formulations for Alleviation or Prevention of Menopausal Symptoms”, filed Oct. 24, 2008. The disclosures in the applications listed above are incorporated by reference.

### FIELD OF THE INVENTION

This invention is in the field of pharmaceutical compositions for the treatment and/or prevention of sex hormones-mediated hair loss.

### BACKGROUND OF THE INVENTION

The demographics suggest that we face a devastating increase in the prevalence of Alzheimer's disease (AD), reinforcing the immediate need for basic and translational neuroscience to develop safe and efficacious estrogen therapy (ET) and hormone therapy (HT) regimens for the brain. Of those affected with AD, 68% are female and 32% are male (Brookmeyer et al., 1998 Am J Public Health 88:13372). Because women have a longer life expectancy than men, the absolute number of women with AD exceeds that of men. However, a double danger exists for women. Results of a meta-analysis of seven sex-specific studies concluded that women are 1.5 times more likely to develop AD than age-matched men (Gao et al., 1998 Arch Gen Psychiatry 55:809), which was supported by the Cache County analysis that showed a clear female gender increase in the incidence of AD (Zandi et al., 2002 JAMA 288:21239).

At the turn of the new millennium in the United States, there were nearly 42 million women over the age of 50 years and, of these, more than 31 million women were over the age of 55 years (North American Menopause Society, 2004). Worldwide, there are currently more than 470 million women aged 50 years or older, and 30% of those are projected to live into their 80s (North American Menopause Society, 2004). These women can anticipate spending one-third to one-half of their lifetime in the menopausal state. Reports on the prevalence of AD vary, but of the 18 million American women in their mid to late 70s, as many as 5 million may suffer from AD, and this figure increases dramatically at older ages (Brookmeyer et al., 1998). The projected exponential increase in the prevalence of AD, along with the anticipated impact on families and society, highlights the imperative for developing strategies to prevent or delay the onset of AD sooner rather than later.

The profound disparities between the largely positive basic science findings of gonadal steroidal action in brain and the adverse outcomes of recent ET/HT clinical trials in women who are either aged postmenopausal or postmenopausal with AD, has led to an intense reassessment of gonadal hormone action and the model systems used in basic and clinical science. One key factor that could contribute to the negative results of the Women's Health Initiative Memory Study (“WHIMS”) trial was the advanced age, more than ten years following menopause, at which ET/HT was initiated in women. Data from both basic science analyses and clinical studies indicate a “healthy cell bias” of estrogen action in the neurons/brains, suggesting that ET/HT acts as an effective preventative therapeutic strategy for age-related cognitive decline and neurodegenerative disorders, such as Alzheimer's disease (“AD”), while it is not an effective treatment strategy. The current widely prescribed ET, conjugated equine estrogens (“CEE”), is a highly complex ET with over 200 different components. Whether CEE provides the optimal therapeutic efficacy has been questioned. Another key issue challenging HT is the optimal composition. For example, the use of progestin, and its timing of administration in conjunction with ET, remains to be determined. Moreover, while ET/HT has long been used in postmenopausal women to delay or reverse some of the problems associated with menopause, epidemiological and clinical studies have uncovered potential long-term risks related to this therapy. The recently revealed risks associated with ET/HT have greatly increased interest in the development of estrogen alternatives that promote beneficial effects of estrogen in brain, bone and the cardiovascular system, while not eliciting deleterious effects in other organs, particularly in breast and uterine tissues.

Another phenomenon which is affected by aging is androgenic alopecia. Androgenic apolecia is by far the largest single type of hair loss to affect both men and women. It is estimated that around 30% of Caucasian females are affected by some levels of androgenic apolecia before menopause.

In addition, approximately one-third of menopausal women, including 25 million American women, experience noticeable androgenic hair loss or change in hair growth during menopause.

The female sex hormone, estrogen, is generally recognized as a promoter of scalp hair growth and an inhibitor of hair growth elsewhere on the body. In that respect, estrogen has the opposite effects of the male sex hormone, testosterone, which can be converted to dihydrotestosterone (DHT). Prior to menopause, while estrogen levels are high, the level of DHT being produced in the skin and follicle region is kept low. When women enter menopause, their levels of estrogen decline with the result that testosterone becomes more bioactive and more of it is converted to DHT in hair follicles. This results in a shorter hair growth cycle, finer hair and eventually, general hair loss.

An increase in estrogen levels is proven to be beneficial for alleviating or stopping hair loss. Indeed, estrogen has been prescribed by physicians to treat hair loss in women. However, the excess of estrogen can promote various diseases such as breast cancers, which prevents the use of such a therapy in clinic.

Androgenic alopecia or male pattern baldness (MPB) accounts for more than 95% of hair loss in men. By the age of 35, two-thirds of American men will experience some degree of appreciable hair loss. By the age of 50, approximately 85% of men have significantly thinning hair. Approximately 25% of men who suffer with MPB begin the painful process before they reach the age of 21.

Hair loss in men is also considered as a phenotypic result from an over-production of DHT in hair follicles or over-sensitivity of hair follicles to DHT. MPB is generally characterized with the onset of a receding hairline and thinning crown. Hair in these areas including the temples and mid-anterior scalp appear to be the most sensitive to DHT. This pattern eventually progresses into more apparent baldness throughout the entire top of the scalp, leaving only a rim or “horseshoe” pattern of hair remaining in the more advanced stages of MPB. For some men even this remaining rim of hair can be affected by DHT.

It has been suggested that estrogen may also offer some benefits in treating MPB. However, duet to the feminizing effects of estrogen, use of estrogen in men has been very limited.

Two nuclear receptors for estrogen (ERs), ERα and ERβ, have been identified. In the central nervous system, both ERα and ERβ are expressed in the hippocampus and cortex of rodent and human brains. Previous studies have demonstrated that both ERα and ERβ can equivalently promote neuronal survival by activating estrogen mechanisms of action in rat hippocampal neurons. Increasing evidence indicates that ERβ is a key requirement for activation of mechanisms that underlie estrogen-inducible neuronal morphological plasticity, brain development, and cognition. ERα, on the other hand, is more predominant in mediating the sexual characteristics of estrogen effects in the reproductive organs such as breast and uterus. Taken together, these data establish a potential therapeutic application for ERβ as a pharmacological target to promote memory function and neuronal defense mechanisms against age-related neurodegeneration such as Alzheimer's disease (AD), while avoiding activating untoward estrogenic proliferative effects in the breast and uterus, although this might be at the cost of lower efficacy due to the lack of activation of ERβ in the brain. Other potential therapeutic advantages associated with ERβ include regulation of estrogen vasculoprotective action and development of interventions targeting diseases such as depression, colon cancer, prostate cancer, obesity, leukemia, and infertility. However, a potential disadvantage of an ERβ-selective ligand is the lack of activation of ERα in bone, as ERα has been demonstrated to mediate estrogen regulation of bone density.

In searching for an effective ERβ-selective estrogen alternative replacement therapy for preventing hair loss, promoting neurological function and preventing age-related neurodegeneration, such as AD, in postmenopausal women, it is of particular interest to identify and develop naturally occurring molecules or analogues that potentially have a less toxic profile for long-term administration. It is known that several plant-derived estrogenic molecules (referred to as “phytoestrogens”) bind to ERα and to ERβ subtypes, and some of these molecules possess moderate binding selectivity for ERβ and exert estrogenic effects in multiple tissues.

The therapeutic efficacy of phytoestrogens in the brain remains controversial. On the one hand, when administered singly, phytoestrogens appeared to be moderately neuroprotective (Zhao, et al., Exp. Biol. Med, 227, 509-519 (2002). On the other hand, a recent clinical trial revealed that a soy protein supplement that contains a mixture of phytoestrogens did not show improved cognitive function in postmenopausal women, when treatment was initiated at the age of 60 years or older. (Kreijkamp-Kaspers, et al. JAMA 2004, 292, 65-74). As discussed previously, when started 10 or more years following menopause in postmenopausal women when age-related neuronal reorganization had taken place, ET/HT has no benefit on neural function. Therefore, it can be extrapolated that age and hormonal “history” may also be important factors regulating the actions of phytoestrogens in the brain, as was the case for the WHIMS trials.

Another issue that can substantially impact the efficacy of phytoestrogen mixtures in the brain is the formulation of phytoestrogens. Soy extracts or soy protein supplements generally contain multiple phytoestrogenic molecules, some of which may be ERα-selective agonists, while others may be ERβ-selective agonists, and others may be ineffective in activating either ERα or ERβ but may function as inhibitors of ER binding of those ERα and/or ERβ phytoestrogenic agonists.

ERα and ERβ have a yin/yang relationship in many contexts where one receptor may antagonize the actions of the other (Weihua, et al. FEBS Lett. 2003, 546, 17-24; Gustafsson, J. A. Trends Pharmacol. Sci. 2003, 24, 479-485). Studies confirmed this observation, showing that coadministration of ERα-selective agonist PPT and ERβ-selective agonist DPN was less efficacious than either PPT or DPN alone in protecting hippocampal neurons against excitotoxic insults. These findings indicate that although both ERα and ER contribute to estrogen promotion of neuronal survival, simultaneous activation of both ER subtypes, ERα and ERβ, in the same context may diminish the efficacy. Accordingly, a presumption can be made that, in addition to the ER antagonism, the ineffectiveness of administering a mixture of phytoestrogens (i.e. a soy protein supplement) may also partly come from the antagonizing actions among different phytoestrogens, which may be ERα selective or ERβ selective.

Development of an ERβ-selective phytoestrogen formulation could maximize the therapeutic benefits associated with activation of ERβ in the brain while minimizing the adverse effects associated with the activation of ERα in reproductive tissues. Moreover, selective targeting of ERβ potentially reduces antagonistic actions that may occur in a complex soy-derived preparation. This naturally occurring ideal formulation would have tremendous therapeutic value in promoting neurological function and preventing AD in a population at risk for losing neurological capacity and losing memory function, i.e., postmenopausal women. To date, no such phytoestrogen formulation exists. Thus, there is a need for select phytoestrogen formulation, generally, and particularly, a formulation that functions in the brain.

It is therefore an object of the present invention to provide an ERβ-selective phytoestrogen formulation maximizing the therapeutic benefits associated with activation of ERβ while minimizing the adverse effects associated with the activation of ERα in reproductive tissues.

It is a further object of the invention to provide such a composition wherein the active ingredients are isolated from natural substances.

It is also an object of the invention to provide a composition that functions as estrogen and promotes estrogenic effects such as hair growth without inducing feminizing effects in reproductive tissues, so that it can be safely used in both women and men.

It is further an object of the invention to provide compositions to prevent one or more symptoms associated with perimenopause, menopause, or postmenopause and methods of making and using thereof.

### SUMMARY OF THE INVENTION

Phytoestrogen pharmaceutical compositions and methods of use for treating and/or preventing perimenopausal, menopausal and postmenopausal symptoms, including promoting and/or sustaining neurological health and preventing age-related neurodegenerative diseases, such as AD, have been developed. These phytoestrogen formulations are composed of a number of plant-derived estrogenic molecules and/or their structural analogs and exhibit a binding preference to ERβ over ERα and agonist activity in non-reproductive organs including brain. These ERβ-selective phytoestrogen formulations cross the blood-brain-barrier and promote estrogen-associated neurotrophism and neuroprotection mechanisms in the brain, without activating feminizing and proliferative mechanisms in the reproductive tissues, and are therefore devoid of estrogen-associated problematic aspects.

These phytoestrogen formulations are therapeutically useful to both women and men to treat and/or prevent hair loss and prostate hypertrophy or cancer. The compositions are administered enterally, transdermally, transmucosally, intranasally or parenterally. The compositions can be formulated for daily, sustained, delayed or weekly/monthly administration.

### BRIEF DESCRIPTION OF THE DRAWINGS

FIG. 1 shows the chemical Structures of 17β-estradiol and the phytoSERMs genistein, daidzein, equal, and IBSO03569.

FIGS. 2A and 28 show the competition binding curves for ERα (FIG. 2A) and ERβ (FIG. 2B) (molar concentration versus fluorescence polarization (mP)) of progesterone (▪), 17β-estradiol (▴), genistein (G, ▾), daidzein (D, ♦), equal (E, ), IBSO03569 (I, X), G+D (+), G+D+E (*), and G+D+E+I (|).

FIGS. 3A and 3B are graphs of mean TST (° C.) over time in weeks for sham-OVX mice treated with the control diet (FIG. 3A) compared to OVX mice given a phyto-β-SERM diet or a soy extract diet (FIG. 3B). FIGS. 3C and 3D are graphs of mean RT (° C.) over time in weeks for sham-OVX mice treated with the control diet (FIG. 3C) compared to OVX mice given a phyto-β-SERM diet or a soy extract diet (FIG. 3D).

### DETAILED DESCRIPTION OF THE INVENTION

### I. Definitions

“Estrogen Receptor”, as used herein, refers to any protein in the nuclear receptor gene family that binds estrogen, including, but not limited to, any isoforms and variants thereof. Human estrogen receptors include the alpha- and beta-isoforms (referred to herein as “ERα” and “ERβ”).

“Estrogen Receptor Modulator”, as used herein, refers to a compound that can act as an estrogen receptor agonist or antagonist of an estrogen receptor or estrogen receptor isoform having an IC₅₀ or EC₅₀ with respect to ERα, ERβ and/or other estrogen receptor isoforms of no more than about 50 μM as determined using the ERα, and/or ERβ transactivation assay described herein. More typically, estrogen receptor modulators have IC₅₀ or EC₅₀ values (as agonists or antagonists) of not more than about 10 μM. Representative compounds are predicted to exhibit agonist or antagonist activity via an estrogen receptor. Compounds preferably exhibit an antagonist or agonist IC₅₀ or EC₅₀ with respect to ERα and/or ERβ of about 10 μM, more preferably, about 500 nM, even more preferably about 1 nM, and most preferably, about 500 pM, as measured in the ERα and/or ERβ transactivation assays. “IC₅₀” is that concentration of compound which reduces or inhibits the activity of a target (e.g., ERα or ERβ) to half-maximal level. “EC₅₀” is that concentration of compound which provides half-maximum effect.

“Selective Estrogen Receptor Modulator” (or “SERM”), as used herein, refers to a compound that exhibits activity as an agonist or antagonist of an estrogen receptor (e.g., ERα, ERβ or other estrogen receptor isoform) in a tissue-dependent or receptor dependent manner. Thus, as will be apparent to those of skill in the biochemistry, molecular biology and endocrinology arts, compounds that function as SERMs can act as estrogen receptor agonists in some tissues, e.g., bone, brain, and/or cardiovascular, and as antagonists in other tissue types, e.g., the breast and/or uterine tissue.

“Phytoestrogen” refers to a naturally occurring compound of plants, such as soybeans, or plant products, such as whole grain cereals, that acts like estrogen or binds to an estrogen receptor.

As used herein, the term “PhytoSERM” refers to natural source phytoestrogens that preferentially target estrogen receptor beta.

As used herein, the term “analogue” refers to a chemical compound with a structure similar to that of another (reference compound) but differing from it in respect to a particular component, functional group, atom, etc.

As used herein, the term “derivative” refers to compounds which are formed from a parent compound by chemical reaction(s).

“Pharmaceutically acceptable salt”, as used herein, refer to derivatives of the compounds defined by Formula I and II wherein the parent compound is modified by making acid or base salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

Modified release dosage form: A modified release dosage form is one for which the drug release characteristics of time, course and/or location are chosen to accomplish therapeutic or convenience objectives not offered by conventional dosage forms such as solutions, ointments, or promptly dissolving dosage forms. Delayed release, extended release, and pulsatile release dosage forms and their combinations are types of modified release dosage forms.

Delayed release dosage form: A delayed release dosage form is one that releases a drug (or drugs) at a time other than promptly after administration.

Extended release dosage form: An extended release dosage form is one that allows at least a twofold reduction in dosing frequency as compared to the drug presented as a conventional dosage form (e.g. as a solution or prompt drug-releasing, conventional solid dosage form).

Pulsatile release dosage form: A pulsatile release dosage form is one that mimics a multiple dosing profile without repeated dosing and allows at least a twofold reduction in dosing frequency as compared to the drug presented as a conventional dosage form (e.g. as a solution or prompt drug-releasing, conventional solid dosage form). A pulsatile release profile is characterized by a time period of no release (lag time) or reduced release followed by rapid drug release.

### II. Compositions

Compositions containing one or more phytoestrogens are described herein. A number of phytoestrogens have been isolated and identified and additional analogs created, all of which have estrogen receptor binding selectivity. In one embodiment, the composition contains two or more plant-derived estrogenic molecules and/or structural analogues, which possess ERβ-binding selectivity and exhibit neuroprotective activity when administered individually. These compositions are useful for preventing estrogen-deficiency associated symptoms and disorders, particularly age-related cognitive decline and neurodegenerative diseases, such as Alzheimer's disease (“AD”). The compositions are also useful for minimizing or preventing one or more symptoms of menopause including, but not limited to, hot flashes, hair loss/thinning, mood changes, insomnia, fatigue, memory problems, and combinations thereof. Some studies have suggested that ERβ is the predominant ER and the main mediator of estrogen action in human skin and hair follicles. With respect to hair growth, ERβ is believed to play a regulatory role on androgen receptor expression in hair follicles leading to a promoting effect on hair growth. Since estrogen's feminizing effects are mainly mediated by ERα, where ERβ is less involved, ERβ should represent a safer therapeutic target for promoting estrogenic actions, for example, hair growth, without exerting feminizing activities.

The compositions should also be useful to prevent or treat prostate cancer in men.

A. PhytoSERMs

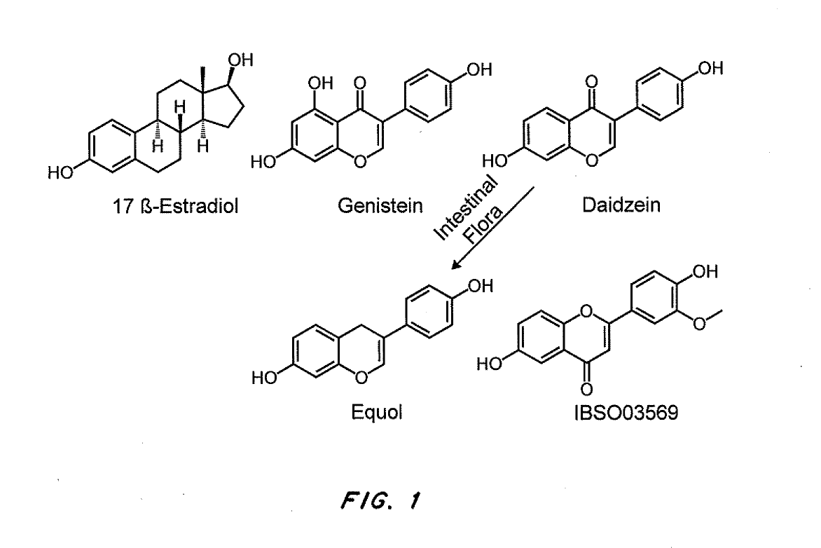

The compositions described herein contain one or more phytoestrogens or natural source selective estrogen receptor modulators (SERMs) exhibiting a binding preference for ERβ. PhytoSERMs can be identified as described in Example 1. Suitable phytoSERMs include, but are not limited to, genistein, daidzein, equol, IBSO03569 and combinations thereof. The structures of genistein, daidzein, equol, and IBSO03569 are shown in FIG. 1. Other potential phytoSERMs are listed in Table 1. Preferred phytoSERMs are those that cross the blood brain barrier. Combinations of two or more PhytoSERMs are more effective than administration of one PhytoSERM.

TABLE 1
Binding Affinity (IC₅₀) and Selectivity of
Representative Molecules for Estrogen Receptor α and β

| | | IC₅₀ | | Selectivity |
|---|---|---|---|---|
| Compd | Structure | ERα | ERβ | (ERα/ERβ) |
| Progesteron | 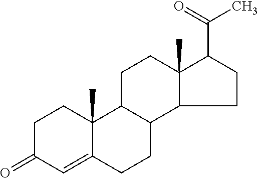 | NC* | NC | |
| genistein | 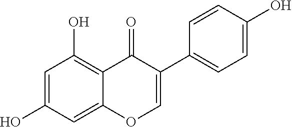 | 4.66 μM | 98.7 nM | 47.2 |
| 1 | 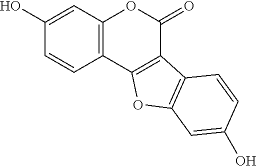 | 75.7 nM | 18.6 nM | 4.07 |
| 2 | 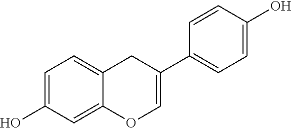 | NC | 0.68 μM | >100 |
| 3 | 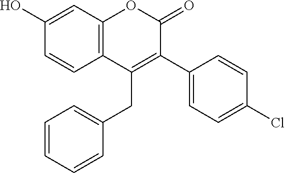 | 120 nM | 250 nM | 0.48 |
| 4 | 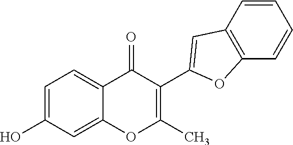 | NC | NC | |
| 5 | 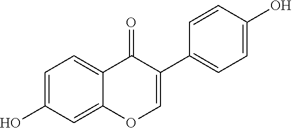 | NC | 2.80 μM | >100 |
| 6 | 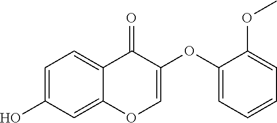 | NC | NC | |
| 7 | 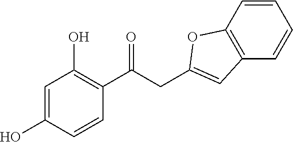 | 85.7 μM | 43.0 μM | 1.99 |
| 8 | 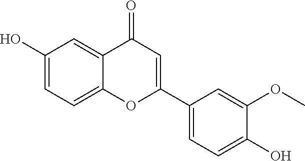 | NC | 4.48 μM | >100 |
| 9 | 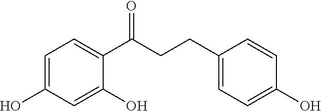 | NC | NC | |
| 10 | 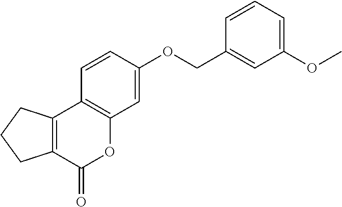 | NC | NC | |
| 11 | 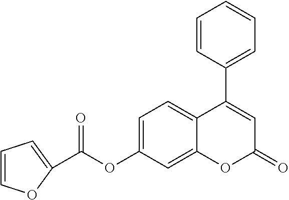 | 2.32 μM | NC | <0.01 |
| 12 | 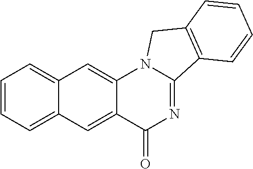 | NC | NC | |

*NC: Nonconvergence within the dose range, predicting that either the molecule does not bind to the receptor or that the biding affinity is very low, with an IC₅₀ greater than 1 mM.

The compounds can be used in the form of salts derived the parent acid or base. The salts can be prepared using organic or inorganic acids or bases. Suitable salts include, but are not limited to, acetate, adipate, alginate, citrate, aspartate, benzoate, benzenesulfonate, bisulfate, butyrate, camphorate, camphorsulfonate, digluconate, cyclopentanepro-pionate, dodecylsulfate, ethanesulfonate, glucoheptanoate, glycerophosphate, hemi-sulfate, heptanoate, hexamate, fumarate, hydrochloride, hydrobromide, hydroiodide, 2-hydroxyethanesulfonate, lactate, maleate, methanesulfonate, nicotinate, 2-napthalenesulfanate, oxalate, pamoate, pectinate, sulfate, 3-phenylpropionate, picrate, pivalate, propionate, succinate, tartrate, thiocyanate, p-toluenesulfonate and undecanoate. Also, any basic nitrogen-containing groups can be quaternized with agents such as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Wafer or oil-soluble or dispersible products are thereby obtained.

Examples of acids which may be employed to form pharmaceutically acceptable acid addition salts include such inorganic acids as hydrochloric acid, sulfuric acid, and phosphoric acid, and organic acids such as oxalic acid, maleic acid, succinic acid and citric acid. Basic addition salts can be prepared in situ during the final isolation and purification of the compounds, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, and aluminum salts, as well as non-toxic ammonium, quaternary ammonium, and mine cations, including, but not limited to ammonium, tetramethylanunonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, and piperazine.

The compounds described herein may have one or more chiral centers and thus exist as one or more stereoisomers. Such stereoisomers can exist as a single enantiomer, a mixture of diastereomers or a racemic mixture.

As used herein, the term “stereoisomers” refers to compounds made up of the same atoms having the same bond order but having different three-dimensional arrangements of atoms which are not interchangeable. The three-dimensional structures are called configurations. As used herein, the term “enantiomers” refers to two stereoisomers which are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

In one embodiment, the composition contains equol and at least one other phytoSERM. The structure of equol is shown below:

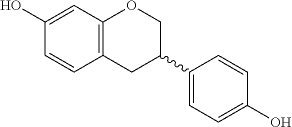

The pyran ring in equol has a chiral center. Therefore, equol can exist as two stereoisomeric forms, which are shown below;

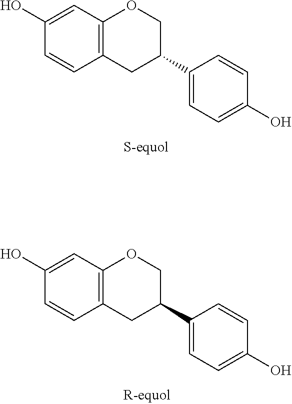

The relative binding affinities of R-equol and S-equol for ERα are 210.6 and 49.2 fold less, respectively, compared to 17β-estradiol. However, the S-equol enantiomer seems to be largely ER-selective with a relatively high affinity for ERβ. It has been reported that the binding affinity of S-equol for ERα is 10-fold less than ERβ. S-equol binds ERβ at approximately 20% that of 17β-estradiol [equal, Kd=0.7 nM vs. 17β-estradiol, Kd=0.15 nM], while the R-equol enantiomer binds at approximately 100 fold less. R-Equol, although not naturally occurring, is of considerable importance because of its ability to modulate androgen-mediated processes in the body.

In one embodiment, the composition contains only the S-enantiomer of equal, only the R-enantiomer of equol, a racemic mixture of the two enantiomers or an enantiomerically enriched mixture of the two enantiomers.

In a preferred embodiment, the compositions contain the R-enantiomer of equol, either alone or in combination with the S-enantiomer. In a more preferred embodiment, the composition contains only the R-enantiomer.

In another preferred embodiment, the compositions contain the S-enantiomer of equol, either alone or in combination with the R-enantiomer. In a more preferred embodiment, the composition contains only the S-enantiomer. S-equol is not present in soy but is produced naturally in the gut of certain individuals, particularly Asians, by the bacterial biotransformation of daidzein, a soy isoflavone. Studies have confirmed that equol is present in man and animals only as the S-enantiomer.

B. Additional Active Agents

While the compounds can be administered as the sole active pharmaceutical agent, they can also be used in combination with one or more other compound as described herein, and/or in combination with other agents used in the treatment and/or prevention of estrogen receptor-mediated disorders. Alternatively, the compounds can be administered sequentially with one or more such agents to provide sustained therapeutic and prophylactic effects. Suitable agents include, but are not limited to, other SERMs as well as traditional estrogen agonists and antagonists.

Representative agents useful in combination with the compounds for the treatment of estrogen receptor-mediated disorders include, for example, tamoxifen, 4-hydroxytarnoxifen, raloxifene, toremifene, droloxifene, TAT-59, idoxifene, RU 58,688, EM 139, ICI 164,384, ICI 182,780, clomiphene, MER-25, DES, nafoxidene, CP-336,156, GW5638, LY 139481, LY353581, zuclomiphene, enclomiphene, ethamoxytriphetol, delmadinone acetate, bisphosphonate. Other agents that can be combined with one or more of the compounds include aromatase inhibitors such as, but not limited to, 4-hydroxymdrostenedione, plomestane, exemestane, aminogluethimide, rogletimide, fadrozole, vorozole, letrozole, and anastrozole.

Still other agents useful in combination with the compounds described herein include, but are not limited to antineoplastic agents, such as alkylating agents, antibiotics, hormonal antineoplastics and antimetablites. An example includes the compounds used to treat or prevent osteoporosis. Other ingredients include vitamins, nutritional supplements, anti-oxidant agents, coenzymes, etc.

The additional active agents may generally be employed in therapeutic amounts as indicated in the PHYSICIANS' DESK REFERENCE (PDR) 53rd Edition (2003), or such therapeutically useful amounts as would be known to one of ordinary skill in the art. The compounds and the other therapeutically active agents can be administered at the recommended maximum clinical dosage or at lower doses. Dosage levels of the active compounds in the compositions may be varied to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions that are given at the same time or different times, or the therapeutic agents can be given as a single composition.

C. Pharmaceutical Compositions

The compounds can be combined with one or more pharmaceutically acceptable carriers, additives, and/or excipient for enteral, transdermal, transmucosal, intranasal, or parenteral administration. The compounds can also be administered via a transdermal patch, a depo, vaginally or rectally using a topical carrier such as a gel, lotion, ointment, liposomal formulation, suspension, foam, spray or suppository, via the pulmonary or nasal route, buccally or sublingual via the mucosal membranes of the mouth. The carriers, additives, and/or excipients are all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of coating compositions which may include plasticizers, pigments, colorants, stabilizing agents, and glidants. Delayed release, extended release, and/or pulsatile release dosage formulations may be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides. Additionally, the coating material may contain conventional carriers such as plasticizers, pigments, colorants, glidants, stabilization agents, pore formers and surfactants.

Excipients for oral formulation are known to those skilled in the art, as discussed briefly below, and can be used to provide immediate, sustained, delayed, pulsed release, and combinations thereof. For parenteral administration, the compounds may be dissolved or suspended in saline, sterile water or phosphate buffered saline, or a suitable oil for injection intravenously (iv), intramuscularly (im), subcutaneously (subcu), intrasternal, infusion, or intraperitoneal (ip).

Suitable pharmaceutically acceptable excipients include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-.beta.-cyclodextrin, polyvinylpyrrollidone, low melting waxes, and ion exchange resins, as well as combinations of any two or more thereof. Other suitable pharmaceutically acceptable excipients are described in Remington's Pharmaceutical Sciences, Mack Pub. Co., New Jersey (1991).

Pharmaceutical compositions containing estrogen receptor modulating compounds may be in any form suitable for the intended method of administration, including, for example, a solution, a suspension, or an emulsion. Liquid carriers are typically used in preparing solutions, suspensions, and emulsions. Liquid carriers contemplated for use include, for example, water, saline, pharmaceutically acceptable organic solvent(s), pharmaceutically acceptable oils or fats, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, surfactants, or stabilizers. Suitable organic solvents include, for example, monohydric alcohols, such as ethanol, and polyhydric alcohols, such as glycols. Suitable oils include, for example, soybean oil, coconut oil, olive oil, safflower oil, cottonseed oil. For parenteral administration, the carrier can also be an oily ester such as ethyl oleate, isopropyl myristate. Compositions may also be in the form of microparticles, microcapsules, liposomal encapsulates, as well as combinations of any two or more thereof.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The compounds may be administered orally, parenterally, sublingually, by inhalation spray, rectally, vaginally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Injectable preparations, for example, sterile injectable aqueous or oleaginous suspensions may be form fated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water; Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid can be useful in the preparation of injectables.

Suppositories for rectal or vaginal administration of the drug can be prepared by mixing the drug with a suitable nonirritating excipient such as cocoa butter and polyethylene glycols that are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifing and suspending agents, cyclodextrins, and sweetening, flavoring, and perfuming agents.

The compounds can also be administered in the form of lipsomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound, stabilizers, preservatives, excipients. The preferred lipids are the phospholipids and phosphatidyl cholines (lecithins), both natural and synthetic. Methods to form liposomes are known in the art (Prescott 1976).

Transdermal patches are well known for delivery of nicotine, nitroglycerin and birth control. These can be utilized with these formulations as well. Depos that are implanted under the skin or ip can also be used, similarly to the manner of delivering birth control.

Appropriate carriers can be incorporated that assist the compounds to cross the blood-brain-barrier.

Modified Release Dosage Forms

The compounds can also be formulated for modified release, such as delayed release, sustained release, pulsatile release, and combinations thereof.

Extended Release Dosage Forms

The extended release formulations are generally prepared as diffusion or osmotic systems, for example, as described in “Remington—The science and practice of pharmacy” (20th ed., Lippincott Williams & Wilkins, Baltimore, Md., 2000). A diffusion system typically consists of two types of devices, a reservoir and a matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer poly(methyl methacrylate), poly(methacrylic acid) (anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename Eudragit®. In further preferred embodiments, the acrylic polymer comprises a mixture of two acrylic resin lacquers commercially available from Rohm. Pharma under the tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. Edragit® S-100 and Eudragit® L-100 are also preferred. The code designations RL (high permeability) and RS (low permeability) refer to the permeability properties of these agents. Eudragit® RL/RS mixtures are insoluble in water and in digestive fluids. However, multiparticulate systems formed to include the same are swellable and permeable in aqueous solutions and digestive fluids.

The polymers described above such as Eudragit® RL/RS may be mixed together in any desired ratio in order to ultimately obtain a sustained-release formulation having a desirable dissolution profile. Desirable sustained-release multiparticulate systems may be obtained, for instance, from 100% Eudragit® RL, 50% Eudragit® RL and 50% Eudragit® RS, and 10% Eudragit® RL and 90% Eudragit® RS. One skilled in the art will recognize that other acrylic polymers may also be used, such as, for example, Eudragit® L.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In the congealing method, the drug is mixed with a wax material and either spray-congealed or congealed and screened and processed.

Delayed Release Dosage Forms

Delayed release formulations are created by coating a solid dosage form with a polymer film, which is insoluble in the acidic environment of the stomach, and soluble in the neutral environment of the small intestine.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

Pulsatile Release

The formulation can provide pulsatile delivery of the one or more neuro-protective agents. By “pulsatile” is meant that a plurality of drug doses are released at spaced apart intervals of time. Generally, upon ingestion of the dosage form, release of the initial dose is substantially immediate, i.e., the first drug release “pulse” occurs within about one hour of ingestion. This initial pulse is followed by a first time interval (lag time) during which very little or no drug is released from the dosage form, after which a second dose is then released. Similarly, a second nearly drug release-free interval between the second and third drug release pulses may be designed. The duration of the nearly drug release-free time interval will vary depending upon the dosage form design e.g., a twice daily dosing profile, a three times daily dosing profile, etc. For dosage forms providing a twice daily dosage profile, the nearly drug release-free interval has a duration of approximately 3 hours to 14 hours between the first and second dose. For dosage forms providing a three times daily profile, the nearly drug release-free interval has a duration of approximately 2 hours to 8 hours between each of the three doses.

In one embodiment, the pulsatile release profile is achieved with dosage forms that are closed and preferably sealed capsules housing at least two drug-containing “dosage units” wherein each dosage unit within the capsule provides a different drug release profile. Control of the delayed release dosage unit(s) is accomplished by a controlled release polymer coating on the dosage unit, or by incorporation of the active agent in a controlled release polymer matrix. Each dosage unit may comprise a compressed or molded tablet, wherein each tablet within the capsule provides a different drug release profile. For dosage forms mimicking a twice a day dosing profile, a first tablet releases drug substantially immediately following ingestion of the dosage form, while a second tablet releases drug approximately 3 hours to less than 14 hours following ingestion of the dosage form. For dosage forms mimicking a three times daily dosing profile, a first tablet releases drug substantially immediately following ingestion of the dosage form, a second tablet releases drug approximately 3 hours to less than 10 hours following ingestion of the dosage form, and the third tablet releases drug at least 5 hours to approximately 18 hours following ingestion of the dosage form. It is possible that the dosage form includes more than three tablets. While the dosage form will not generally include more than a third tablet, dosage forms housing more than three tablets can be utilized.

Alternatively, each dosage unit in the capsule may comprise a plurality of drug-containing beads, granules or particles. As is known in the art, drug-containing “beads” refer to beads made with drug and one or more excipients or polymers. Drug-containing beads can be produced by applying drug to an inert support, e.g., inert sugar beads coated with drug or by creating a “core” comprising both drug and one or more excipients. As is also known, drug-containing “granules” and “particles” comprise drug particles that may or may not include one or more additional excipients or polymers. In contrast to drug-containing beads, granules and particles do not contain an inert support. Granules generally comprise drug particles and require further processing. Generally, particles are smaller than granules, and are not further processed. Although beads, granules and particles may be formulated to provide immediate release, beads and granules are generally employed to provide delayed release.

### III. Methods of Administration

Compounds can be administered in a variety of ways including enteral, parenteral, pulmonary, nasal, mucosal and other topical or local routes of administration. For example, suitable modes of administration include oral, subcutaneous, transdermal, transmucosal, iontophotetic, intravenous, intramuscular, intraperitoneal, intranasal, subdural, rectal, vaginal and inhalation. In one embodiment, the compounds are administered topically to an area in which hair growth is desired. This may be in the form of a shampoo, ointment, gel or lotion, optionally in combination with a transdermal penetration enhancer, such as an alcohol, DMSO, and/or surfactants

An effective amount of the compound or composition is administered to treat and/or prevent an estrogen receptor-mediated disorder in a human or animal subject. Modulation of estrogen receptor activity results in a detectable suppression or up-regulation of estrogen receptor activity either as compared to a control or as compared to expected estrogen receptor activity. Effective amounts of the compounds generally include any amount sufficient to detectably modulate estrogen receptor activity by any of the assays described herein, by other activity assays known to those having ordinary skill in the art, or by detecting prevention and/or alleviation of symptoms in a subject afflicted with an estrogen receptor-mediated disorder.

The amount of active ingredient that may be combined with the carrier materials to produce a single dosage form will vary depending upon the estrogen-mediated disease, the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The prophylactically or therapeutically effective amount for a given situation can be readily determined by routine experimentation and is within the skill and judgment of the ordinary clinician. In the case of hair loss or prostate disease, an effective amount is that which stops or decreases further hair loss, or which restores hair growth in areas where it is decreased. In the case of prostate overproliferation, for example, benign prostate hypertrophy or prostate cancer, an effective amount is that which decreases proliferation of cells associated with the prostate gland in men.

For exemplary purposes, a prophylactically or therapeutically effective dose of the phytoestrogens is from about 0.01 mg/kg/day to about 20 mg/kg/day, more preferably from about 0.05 mg/kg/day to about 10 mg/kg/day, most preferably from about 0.1 mg/kg/day to about 5 mg/kg/day, wherein the formulation is administered daily in a single dose or divided doses.

Estrogen receptor-mediated disorders that may be treated include any biological or medical disorder in which estrogen receptor activity is implicated or in which the inhibition of estrogen receptor potentiates or retards signaling through a pathway that is characteristically defective in the disease to be treated. The condition or disorder may either be caused or characterized by abnormal estrogen receptor activity. Representative estrogen receptor-mediated disorders include, for example, osteoporosis, atherosclerosis, estrogen-mediated cancers (e.g., breast and endometrial cancer), Turner's syndrome, benign prostate hyperplasia (i.e., prostate enlargement), prostate cancer, elevated cholesterol, restenosis, endometriosis, uterine fibroid disease, hot flashes, and skin and/or vagina atrophy. The compositions may also be used to treat one or more symptoms associate with the various stages of menopause including, but not limited to, hot flashes, hot flushes, hair loss/thinning, mood changes, insomnia, fatigue, memory problems, and combinations thereof. The composition may also be useful in treating hair loss/thinning in men.

Successful treatment of a subject may result in the prevention, inducement of a reduction in, or alleviation of symptoms in a subject afflicted with an estrogen receptor-mediated medical or biological disorder. Thus, for example, treatment can result in a reduction in breast or endometrial tumors and/or various clinical markers associated with such cancers. Treatment of Alzheimer's disease can result in a reduction in rate of disease progression, detected, for example, by measuring a reduction in the rate of increase of dementia.

### IV. Kits

Kits may be provided which contain the formulation to be administered. The formulation may be administered once a day or more than once a day. The formulation can be administered enterally, parenterally, or topically. The kits typically contain the active agent(s) to be administered, excipients and carriers, and instructions for administration of the formulation. The kits may also contain equipment/devices used to administer the formulation, such as syringes.

The present invention will be further understood by reference to the following non-limiting examples.

### EXAMPLES

### Example 1

### Identification of PhytoSERMs

ERβhas been associated with estrogen-induced promotion of memory function and neuronal survival. Based on the optimized complex structure of human ERβ LBD bound with genistein, computer-aided structure-based virtual screening against a natural source chemical database was conducted to determine the occurrence of plant-based ERβ-selective ligands. Twelve representative hits derived from database screening were assessed for their binding profiles to both ERs, three of which displayed over 100-fold binding selectivity to ERβ over ERα.

Materials and Methods

Identification of Compounds in Database

The ligand binding domains of the human ERα and ERβ are approximately 60% homologous. Structural modeling and mutational analyses indicate that two variant amino acid residues along the ligand binding pocket, Leu 384 and Met 421 in ERα, which are replaced with Met 336 and Ile 373, respectively, in ERβ, are the key molecular constituents underlying discriminative binding of selective ligands to either receptor subtypes. Sun, et al. Mol. Endocrinol. 2003, 17, 247-258. This slight structural variance serves as the foundation for both design and discovery of ER specific ligands. The similarities in the chemical features of both pairs of residues presents a substantial challenge to discover a selective ligand based on this difference. Of the known natural source ERβ-selective ligands, genistein remains the most selective. However, an increasing number of synthetic compounds are emerging showing greater selectivity than genistein for ERβ, as evidenced by the compound DPN developed in Katzellenebogen's laboratory. Computer-aided structure-based virtual database screening provides an efficient approach to rationally highlight a small group of lead candidates from a large number of compounds for investigation at the bench.

All computational work was performed on a SGI Octane workstation equipped with the IRIX 6.5 operating system (Silicon Graphic Inc.). First, the 3D crystallographic structure of human ERβ LBD complexed with genistein was downloaded from the Protein Data Bank (PDB ID: 1QKM). The complex structure was fixed and energy minimized with the Accelrys molecular modeling software package InsightII 2000 (Acceirys Inc.). An in-house 2D natural source chemical collection containing approximately 25 000 plant-based natural molecules or derivatives was converted to a 3D multiconformational database with the Accelrys modeling software package Catalyst 9.8 (Acceirys Inc.).

The receptor-docking site was defined based on the binding position of genistein in the receptor and specified as all atoms within 10 Å of the center carbon of genistein. GOLD 2.0 (Genetic Optimization for Ligand Docking), an automated ligand docking program distributed by CCDC (Cambridge Crystallographic Data Center), was applied to calculate and rank the molecules based on their complementarities with the receptor binding site, on both geometrical and chemical features.

Prior to the database screening, initial validation using genistein as the test ligand was conducted. The aim of the validation test was to evaluate the effectiveness of the algorithm of the docking program in identifying the experimentally observed binding mode of the ligand in the receptor, to determine whether the program is applicable to the specific target system in the examples. In addition, the validation test was used to determine the optimal parameter settings for the later database screening. Twenty docking runs were carried out on the test complex, using the fastest default generic algorithm parameters optimized for virtual library screening, and the GoldScore fitness function was applied. The validation test demonstrated that, based on the specified parameter settings, GOLD was effective in capturing the contributive hydrogen bond donor (ND1 in His 475) crucial to the binding and reproducing the nearly coincident solution in terms of both the binding orientation and conformation of genistein as observed in the experimental measurement (see FIG. 1). The root-mean-square (RMS) deviations were computed between the observed experimental position and the GOLD solutions, with RMSD 0.3299 and 0.4483 compared to top-ranked and worst solutions, respectively. The average RMSD of all solutions was 0.3566, which is regarded as a good prediction based on the subjective classifications defined by the program developer (refer to the program manual), suggesting that this program is reliable and applicable to the database screening toward ERβ.

Using the parameter settings determined in the validation test, the 3D natural source chemical database was input and docked into the prepared ERβ binding site in a flexible docking manner (full ligand and partial protein) and scored based on the GoldScore fitness function. Five hundred resultant top-scoring molecules were filtered via visual screening in the context of the receptor in InsightII. Based on visual analysis, 100 molecules underwent further analysis by Affinity, a more complex and predictive ligand docking program to refine the binding modes predicted by GOLD. The criteria used for the selection of candidate molecules for investigation included the following (a) formation of hydrogen bond with donor atom ND1 in His 475; (b) hydrophobic and hydrophilic balance appearing in the structure (e.g., the molecule should potentially have two relatively hydrophilic sides and a hydrophobic center to enhance both the steric and electrostatic complementarity with the receptor); (c) bound pose of the molecule in the receptor; and d) structural diversity. Finally, molecules that met the above criteria were computationally predicted for their drug-likeness (Lipinski's Rule of Five) and blood-brain bather (BBB) penetration properties.

Determination of Binding Affinity and Selectivity

The binding affinity and selectivity of candidate molecules yielded from database screening were determined by a fluorescent polarization competitive binding assay using purified baculovirus-expressed human ERβ or ERα and a fluorescent estrogen ligand EL Red (PanVera Corp.). Test molecules were serially diluted to a 2× concentration in assay buffer (200 μM to 200 pM). Fifty microliters of preincubated 2× complex of ERβ (30 nM) or ERβ (60 nM) and EL Red (2 nM) was added to each well in a 96-well Non-binding Surface black microplate (Corning Life Sciences) for a final volume of 80 or 100 μL. Negative controls containing ER and EL Red (equivalent to 0% inhibition) and positive controls containing only free EL Red (equivalent to 100% inhibition) were included. After a 2 hour or 6 hour incubation period at room temperature, the polarization values were measured using a Tecan GENios Pro reader at 535 nm/590 nm excitation/emission and plotted against the logarithm of the test molecule concentration. IC₅₀ values (concentration of test molecule that displaces half of the EL Red from ER) were determined from the plot using a nonlinear least-squares analysis available from GraphPad Prism Version 4.03 (GraphPad Software, San Diego, Calif.).

Results

31 molecules that can form a hydrogen bond with ND1 in His 475 were selected and grouped into three categories based upon the chemical features that favored both the van der Waals (VDW) contact (the number of the rings in the structure) and electrostatic interactions (the number of the hydrogen bonds) with the receptor. 10 molecules that have strong VDW interactions with the receptor, but without contributive hydrogen bonding, were grouped in Category IV. These molecules contain three or four five- or six-membered rings in their structures that could promote the hydrophobic interactions with the center of the receptor binding site as observed in endogenous estrogen 17β-estradiol that consists of four rings in its structure and binds to the estrogen receptor with a high affinity.

Table 1 summarizes the IC₅₀ binding results of test molecules to both ERα and ERβ as well as the binding selectivity of representative molecules selected from four categories.

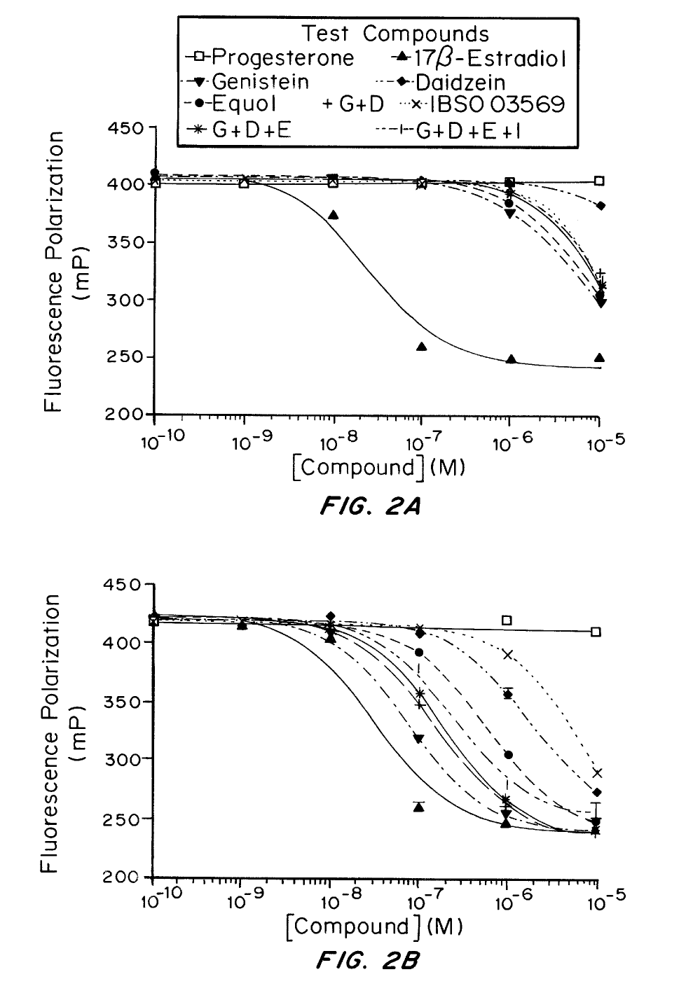

FIGS. 2A and 2B show the competition binding curves for ERα (FIG. 2A) and ERβ (FIG. 2B) (molar concentration versus fluorescence polarization (mP)) of progesterone (▪), 17β-estradiol (▴), genistein (G, ▾), daidzein (D, ♦), equol (E, ), IBSO03569 (I, X), G+D (+), G+D+E (*), and G+D+E+I (|).

TABLE 1
Binding Affinity (IC₅₀) and Selectivity of
Representative Molecules for Estrogen Receptor α and β

| | | IC₅₀ | | Selectivity |
|---|---|---|---|---|
| Compd | Structure | ERα | ERβ | (ERα/ERβ) |
| Progesteron | 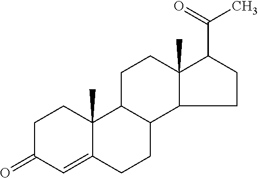 | NC* | NC | |
| genistein | 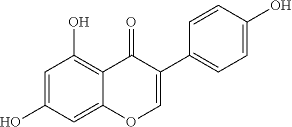 | 4.66 μM | 98.7 nM | 47.2 |
| 1 | 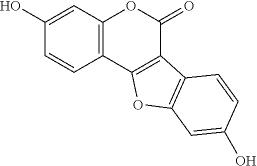 | 75.7 nM | 18.6 nM | 4.07 |
| 2 | 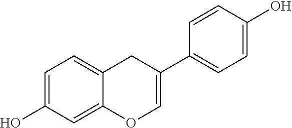 | NC | 0.68 μM | >100 |
| 3 | 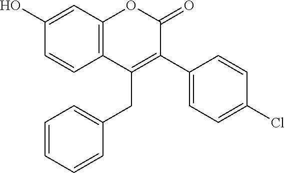 | 120 nM | 250 nM | 0.48 |
| 4 | 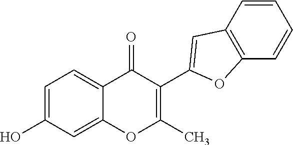 | NC | NC | |
| 5 | 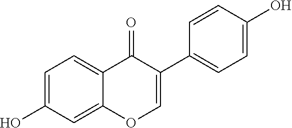 | NC | 2.80 μM | >100 |
| 6 | 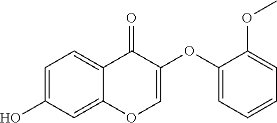 | NC | NC | |
| 7 | 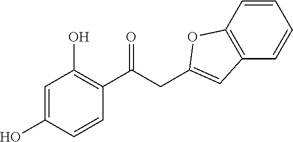 | 85.7 μM | 43.0 μM | 1.99 |
| 8 | 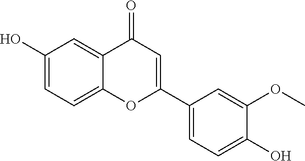 | NC | 4.48 μM | >100 |
| 9 | 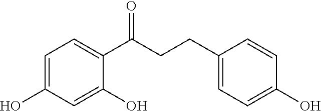 | NC | NC | |
| 10 | 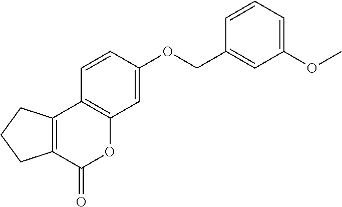 | NC | NC | |
| 11 | 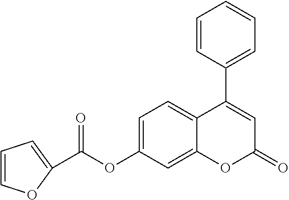 | 2.32 μM | NC | <0.01 |
| 12 | 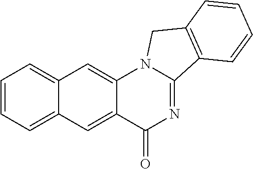 | NC | NC | |

*NC: Nonconvergence within the dose range, predicting that either the molecule does not bind to the receptor or that the biding affinity is very low, with an IC₅₀ greater than 1 mM.

As expected, the negative control steroid, progesterone, does not bind to either ER. As a positive natural source estrogen control, genistein was found to bind to ERβ with a 47.2-fold greater binding selectivity over ERα, but at an affinity one-fourth of 17β-estradiol. Among 12 molecules tested, five molecules, 1, 2, 5, 7, and 8, showed binding selectivity to ERβ over ERα, 3 of which, 2, 5, and 8, displayed the selectivity over 100-fold. Preliminary structure and binding activity relationship analyses revealed that both the central hydrophobic skeletal structure and the connected two polar ‘arms’ contribute to the binding affinity of ligands to both ERs. The enhanced VDW contact derives mainly from the central hydrophobic feature of the molecule. For example, the number of rings increases the binding affinity of molecules to the receptor, as indicated by the VDW value of 17β-estradiol (−67.98) versus that of genistein (−60.75) and molecule 9 (−58.04), which are well correlated with their order-different binding affinities. Meanwhile, the hydrogen bonds derived from the two polar “arms” of the molecule are essential for the binding as well. The lack of one “arm” of the hydrogen bond, as represented by molecule 4 and 6, or two ‘arms’, as represented by 10 and 12, even though the latter two molecules can elicit strong VDW interactions with the receptor, with the VDW value of −72.58 and −69.19, respectively, leads to either very weak or no binding. With respect to the binding selectivity, as demonstrated in the modeling complex structures of a synthetic ERβ-selective agonist, PPT, Stauffer, et al. J. Med. Chem. 2000, 43, 4934-4947 and a synthetic ERβ-selective agonist, DPN, Meyers, et al., J. Med. Chem. 2001, 44, 4230-4251, with both ERs, Zhao, et al. 2004 Abstract Book, The Keystone Symposia: Nuclear Receptors: Steroid Sisters, Keystone, Colo. February 2004, relatively larger molecular size favors the binding selectivity for ERβ over ERα, as represented by molecule 3 and 11.

3 out of 12 representative molecules yielded from database searching displayed over 100-fold selectivity toward ERβ over ERα, demonstrating the effectiveness of this computer-aided virtual screening approach applied in the examples in the discovery of potential molecules that preferentially interact with ERβ.

### Example 2

### A phytoSERM Formulation that Selectively Binds ERβ Prevents Ovariectomy (OVX)-Induced Hair Thinning in Mice

Methods and Materials

Custom diets. Three rodent diets were custom manufactured by Harlan Laboratories (Madison, Wis.). The Control Diet, which also served as the base diet for the other two diets, was prepared from Teklad Global 16% Protein Rodent Diet (Harlan Laboratories), which was ground and re-pelleted. This diet has a fixed formula and is nutritionally balanced containing 16% protein and 3.6% fat that support the growth and maintenance of rodents. This diet does not contain alfalfa or soybean meal, thus minimizing the occurrence of natural phytoestrogens. The Phyto-β-SERM Diet was prepared by adding equal parts of genistein, daidzein and equol (LC Laboratories, Woburn, Mass.), 0.0333 g/kg each, to the base diet. Total addition sums to approximately 100 mg (genistein, daidzein and equol)/kg diet. This diet delivers 10 mg added phyto-β-SERM formulation/kg mouse/day, assuming a 25 g mouse eating 2.5 g/day. The Soy Extract Diet was prepared by adding a commercial soy extract product, Healthy Women® Soy Extract Supplement (Amazon, Seattle, Wash.), to the base diet. Total addition sums to approximately 100 mg (genistein/genistin, daidzeinldaidzin and glycitein/glycitin)/kg diet. This diet would deliver 10 mg added total phytoestrogens/kg mouse/day, assuming a 25 g mouse eating 2.5 g/day.

The phytoestrogen-enriched phyto-β-SERM and soy extract diets were designed to deliver to mice a total amount of phytoestrogens biologically equivalent to a daily intake of 50 mg in humans. This human dose is the estimated average amount of phytoestrogens that Asians regularly ingest from dietary consumption of soy foods, it is also the recommended daily serving dose for many commercial soy extract supplements sold to women in the US, including the one tested in this study.

Animals and treatment. The use of animals and treatment were approved by the Institutional Animal Care and Use Committee at the University of Southern California. Two separate studies were conducted in which adult female 129/C57BL/6 mice were ovariectomized (OVX) or underwent a sham operation and immediately fed one of the three custom diets prepared above. The two-month treatment study, starting in 6-month-old mice, was designed to evaluate the thermal regulation of the diets. Throughout the treatment, mouse tail skin temperature (TST) and rectal temperature (RT) were recorded every other day. The 9-month treatment study, starting in 3-month-old mice, was designed to evaluate the long-term impact of the diets on both the physical and neurological changes associated with menopause. Throughout the treatment, mouse food intake and body weigh were recorded, and physical appearances were photographed, 1-2 times a week. After 8.5-month treatment, 2 weeks before the treatment was ended, a cognition-behavioral test of spatial working memory function, Y-Maze, was administered. At the time of sacrifice, uteri were excised, trimmed of fat and connective tissue, and a wet weight was recorded. Brain tissues were collected and dissected into hippocampus, cortex and cerebellum. Hippocampus were further processed into protein samples and analyzed by Western blot for changes in expression levels of proteins associated with learning and memory.
TST and RT measurement. TST and RT were recorded in a temperature-controlled test room with the experimenter blind to treatment conditions. Mice were first acclimated to handling and experimental apparatuses over a period of 2 weeks. Recordings were performed at 1500 h of the light phase of the light/dark cycle. TST was recorded with a small rodent infrared thermometer (Model No. IR-B152, Braintree Scientific, Braintree, Mass. 02185), which was placed at the dorsal surface of the tail approximately 1 cm from the base of the tail. Over the course of 2 min, four readings at a 30-sec interval were recorded and the average of the last two readings was reported as the final TST. Following the TST measurement, RT was recorded with an animal rectal probe designed for use in mice (Model No. RET-3, Braintree Scientific) attached to a MicroTherma 2 type “T” thermometer (Model No. TW2-193, Braintree Scientific).
Y-Maze cognition-behavioral test. Y-Maze with three identical arms that are evenly spaced with an arm length of 35 cm, arm height of 10 cm and lane width of 5 cm (Model No. 60180, Stoelting, Wood Dale, Ill.), was used. Test was conducted in a temperature-controlled test room with accurate configuration of spatial visual cues. The experimenter was blind to treatment conditions. In the one-trial test of spontaneous alteration behavior (SAB), mice were allowed to move freely within the Maze for 5 min. The total number and the order of arm entries were recorded. Alteration is defined as successive entries into the three arms in overlapping triplet sets. The percent alteration is calculated as the ratio of actual to possible alterations: (the total number of arm entries−2)×100. The two-trial recognition test consisted of two trials separated by an inter-trial interval. In the first acquisition trial, one arm of the Maze was closed and mice were allowed to explore the two other arms for 10 min. During the 5 h of interval, mice were housed in their home cages located in a room other than the test room. In the second retention trial, mice had free access to all three arms, and were allowed to explore the Maze for 5 min. The first arm entered, the number of entries into each arm and the amount of time spent in the novel arm were recorded. The number of visits in the novel arm is calculated as a percentage of the total number of visits in all three arms during the first 2 min of the second trial.
Western blot. Hippocampal tissue-derived protein extraction and concentration measurement were performed. 20-40 μg of protein samples were loaded per lane and separated by electrophoresis on a 10-12% SDS-PAGE. Proteins were then electrotransferred to PVDF membranes and probed with primary antibodies against brain-derived neurotrophic factor (BDNF; 1:250, Chemicon, Temecula, Calif.), synaptophysin (SYP; 1:8000, Millipore, Billerica, Mass.), postsynaptic density protein 95 (PSD-95; 1:1000, Chemicon), apolipoprotein (ApoE; 1:2000, Chemicon), insulin-degrading enzyme (IDE; 1:1000, Calbiochem, San Diego, Calif.), and neprilysin (NEP; 1:250, Chemicon), at 4° C. overnight, and then with HRP-conjugated secondary antibodies (1:5000-20000, Vector Laboratories, Burlingame, Calif.). β-tubulin (1: 5000, Abeam, Cambridge, Mass.) was used as the loading control. Bands were visualized with a TMB peroxidase kit (Vector Laboratories) or by chemiluminescence using an ECL detection kit (Amersham, Piscataway, N.J.). Relative intensities of the immunoreactive bands were captured by Molecular Imager ChemiDoc XRS+System (Model No. 170-8251, Bio-Rad, Hercules, Calif.) and quantitated by Quantity One Analysis Software, Version 4.6.4 (Model No. 170-9600, Bio-Rad).
Statistical analyses. Data are presented as group means±S.E.M. Statistically significant differences were determined by a one-way analysis of variance followed by a Student-Newman-Keuls post hoc analysis.

Results

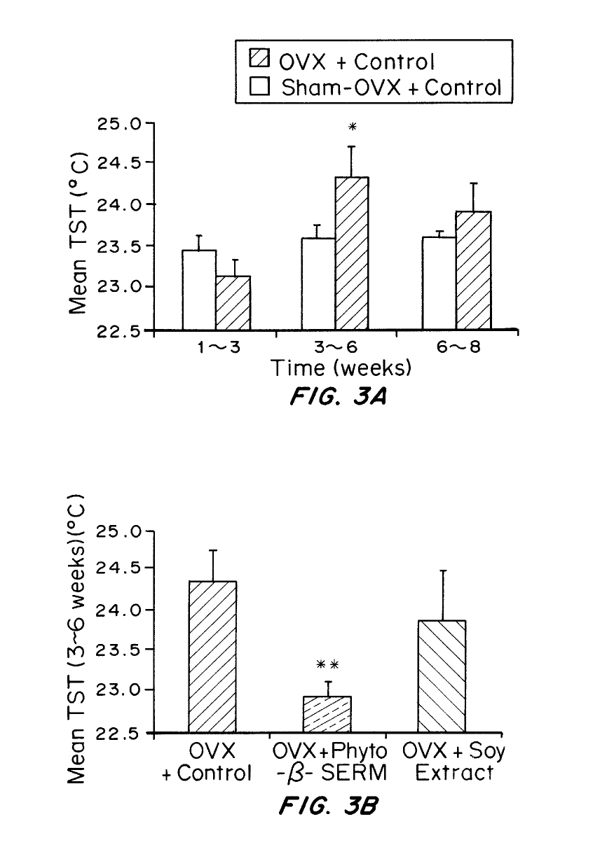

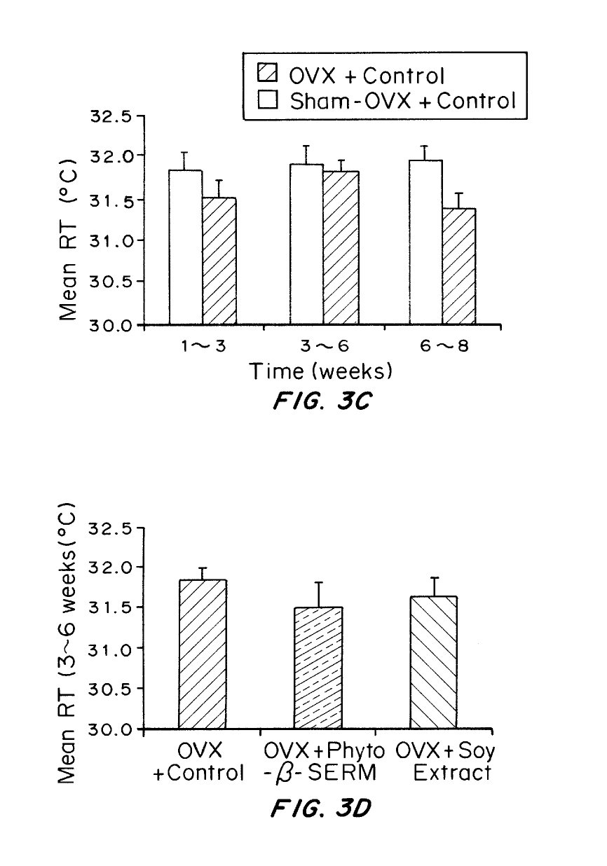

Phyto-β-SERM diet, not soy extract diet, prevented OVX-induced rise in TST. The first 2-month experiment was designed to investigate whether the phyto-β-SERM formulation would have efficacy to mitigate the most common menopausal symptom, hot flashes. Following an OVX or sham operation, 6-month-old female mice were fed immediately one of the three test diets and the treatment lasted for 8 weeks. Data shown in FIG. 3a revealed that when compared to sham-OVX mice treated with the control diet, OVX mice under the same diet exhibited a significant rise in TST, the surrogate marker of hot flashes, at 3-6 weeks following OVX (FIG. 3a; * P<0.05 compared to 1-3 weeks). OVX-induced rise in TST at 3-6 weeks was prevented by the phyto-β-SERM diet, but not by the soy extract diet (FIG. 3b; ** P<0.01 compared to OVX mice treated with the control diet). In comparison with TST, RT, the indicator of the core body temperature, did not show significant changes with either the estrogen (FIG. 3c) or dietary status (FIG. 3d) throughout the same observational period (3-6 weeks).
Phyto-β-SERM diet, not soy extract diet, prevented OVX-induced abnormalities in hair growth. The following 9-month experiment was designed to investigate the long-term impact of the phyto-β-SERM formulation on the physical appearance and neurological functional changes associated with menopause. Following an OVX or sham-OVX operation, 3-month-old female mice were fed immediately one of the three test diets and the treatment lasted for 9 months. Although there appeared a positive effect from the two phytoestrogen-supplemented diets on the growth of mice, none of the three test diets induced a significant change in the body weight of treated mice. However, mice fed different diets exhibited profound differences in their physical appearances. When compared with sham-OVX mice treated with the control diet, OVX mice treated with the same diet exhibited an abnormal hair thinning/loss which were particularly noted around the forehead and neck. Such an abnormality in hair growth appeared even worse in OVX mice treated with the soy extract diet. In a stark contrast, OVX mice treated with the phyto-β-SERM diet looked no different from sham-OVX mice under the control diet.

### SUMMARY OF RESULTS

The first experiment, started in 6-month-old mice and lasted for 8 weeks, was designed to investigate the therapeutic potential of the dietary treatment on menopausal hot flashes, the most complained-about climacteric symptom that occurs in nearly 80% of Caucasian women during menopause. Clinically, menopausal hot flashes manifest as a transient increase in skin temperature and profuse sweating, resulted primarily from the gradual cessation of the ovarian estrogen production at menopause. In preclinical research, the elevation of TST in OVX mice or rats has been widely used as a genuine experimental model to simulate menopausal hot flashes in women. A time-dependent significant change in TST, but not in RT, was observed in response to OVX or dietary treatment in the mouse model. It was further observed that the phyto-β-SERM diet, but not the soy extract diet, prevented OVX-induced rise in TST throughout the same time course. These observations support the therapeutic potential of the phyto-β-SERM formulation in the intervention of menopausal hot flashes. Soy extract-based products, however, could have no benefit as they are thought.

Encouraged by the positive data on hot flashes, a second experiment with a longer duration, which, started in 3-month-old mice and lasted for 9 months, was designed to investigate the potential impact of the dietary treatment on the general health and neurological function associated with menopause. One significant phenomenon observed from this long-term treatment study was the changes in physical appearance, in particular in hair growth, in relation to OVX or the dietary treatment. In clinic, androgenic hair thinning/loss is another commonly noted climacteric symptom that occurs in approximately one-third of menopausal women, including 25 million American women. An increase in estrogen levels has been proven to be beneficial for alleviating or stopping menopausal hair thinning/loss. Moreover, research indicate that ERβ is the predominant ER and the main mediator of estrogen action in human skin and hair follicles, where ERβmay play a regulatory role on androgen receptor expression leading to an alteration on hair growth. In the present study, the observation of the regional hair thinning/loss around the forehead and neck of OVX mice resembles to a great extent clinical characteristics in menopausal women (http://www.pioneerthinking.com/hairloss.html), indicating that the hair abnormalities observed in these mice are directly associated with estrogen deficiency induced by OVX. OVX mice treated with the phyto-β-SERM diet did not look differently from the sham-OVX control mice. In contrast, OVX mice treated with the soy extract diet appeared similar to or even worse in some mice than OVX control mice. These observations provide further support for the therapeutic potential of the phyto-β-SERM formulation, but not the soy extract, in the intervention of climacteric symptoms, including the change in hair growth.

The phyto-β-SERM formulation did not affect the uterine growth of treated animals, as reported in Zhao, et al., Endocrinology. 2009, 150:770-783. The present observation that a 9-month exposure to the phyto-β-SERM diet did not induce a significant change in uterine weight of treated mice further confirms the lack of estrogenic proliferative property from the phyto-β-SERM formulation, suggesting that different from ET, the phyto-β-SERM formulation does not induce a risk for reproductive cancers.

In addition to the differences in the compositional complexity and related therapeutic effectiveness, the phyto-β-SERM formulation offers two more clinical advantages over soy extract products. The first advantage is associated with the high selectivity for ERβ by the phyto-β-SERM formulation. Numerous studies have indicated that although both ERα and ERβ mediates estrogen-induced neuroprotection (Zhao et al., Brain Res. 2007, 1172:48-59; Zhao, et al., Brain Res. 2004, 1010:22-34), ERβ could be more involved in estrogen regulation of neural development and trophism. An ERβ-selective therapy could also potentially minimize ERα-mediated feminizing and proliferative responses known to cause elevated risks for reproductive cancers in women, therefore, it should be much safer even with a long-term administration than a non-selective, for instance, ET. The second advantage is associated with the presence of equol in the phyto-β-SERM formulation. Unlike genistein and daidzein, equol is not of plant origin, yet can be exclusively produced through the metabolism of daidzein catalyzed by intestinal microbial flora following the intake of soy products. Wide inter-individual variations in equol-producing phenotype exist across human populations. Only about 20-35% of Western adults are equol-producers as compared to 55-60% in Asian populations. Research has suggested that the equol-producing phenotype could serve as a critical modulator of human response to phytoestrogen treatment. In other words, an enhanced response could occur in equol-producers as compared to non-producers. Inclusion of equol in the phyto-β-SERM formulation could potentially benefit both equol-producers and non-producers.

We claim:

1. A method for alleviating or preventing sex hormone-mediated hair loss comprising administering to a person an effective amount of a phytoestrogen formulation that preferentially binds to estrogen receptor β, to prevent or reduce sex hormone-mediated hair loss or enlarged prostate or prostate cancer.

2. The method of claim 1 wherein the phytoestrogen consists of two or more naturally occurring compounds that preferentially bind to estrogen receptor β, in an amount effective to prevent or reduce sex hormones-mediated hair loss in a subject.

3. The method of claim 2, wherein the two or more naturally occurring compounds are selected from the group consisting of genistein, daidzein, racemic equol, purified or enriched S-enantiomer of equol, purified or enriched R-enantiomer of equol, and IBSO03569.

4. The method of claim 1, wherein the formulation is administered to a male human.

5. The method of claim 1 wherein the formulation is administered to a female human.

6. The method of claim 5 wherein the formulation is administered to a peri-menopausal, menopausal or post-menopausal woman.

7. The method of claim 1, further comprising administering one or more additional active agents selected from the group consisting of other selective estrogen receptor modulators, estrogen agonists, estrogen antagonists, vitamins, nutritional supplements, antioxidants and coenzymes.

8. The method of claim 1 wherein the formulation is for enteral, parenteral, or topical administration.

9. The method of claim 1, wherein the dose of the phytoestrogens is from about 0.01 mg/kg/day to about 20 mg/kg/day.

10. The method of claim 9, wherein the dose of the phytoestrogens is from about 0.05 mg/kg/day to about 10 mg/kg/day.

11. The method of claim 10, wherein the dose of the phytoestrogens is from about 0.1 mg/kg/day to about 5 mg/kg/day.

12. The method of claim 1, wherein the formulation is administered in a single dose or divided doses.

13. The method of claim 1, wherein the formulation is for modified release.

14. The method of claim 13, wherein the modified release is selected from the group consisted of sustained release, delayed release, pulsatile release, and combinations thereof.

15. The method of claim 1, wherein the formulation is administered daily, weekly, biweekly, or monthly.

16. A dosage formulation for use in the method of claim 1.

17. A pharmaceutical composition comprising the S-enantiomer of equal, the R-enantiomer of equal or an enantiomerically enriched mixture thereof and one or more additional compounds that preferentially binds to estrogen receptor β.

18. The composition of claim 17, wherein the one or more additional compounds are selected from the group consisting of genistein, daidzein, and IBSO03569.
